(19) United States
(12) Patent Application Publication
(10) Pub. No.: US 2002/0055112 A1
(43) Pub. Date: May 9, 2002

(54) Methods for reducing complexity of nucleic acid samples

(76) Inventors: Nila Patil, Woodside, CA (US); David Cox, Belmont, CA (US)

Correspondence Address: FULBRIGHT & JAWORSKI, LLP, 1301 MCKINNEY, SUITE 5100, HOUSTON, TX, 77010-3095, US

(21) Appl. No.: 09/938,878

(22) Filed: Aug. 24, 2001

Related U.S. Application Data
(60) Provisional application No. 60/228,251, filed on Aug. 26, 2000.

(51) Int. Cl. C12Q 1/68; C12P 19/34

(52) U.S. Cl. 435/6; 435/91.2

(57) ABSTRACT

The invention provides several methods for reducing the complexity of a population of nucleic acids prior to performing an analysis of the nucleic acids on a nucleic acid probe array. The methods result in a subset of the initial population enriched for a desired property, or lacking nucleic acids having an undesired property. The resulting nucleic acids in the subset are then applied to the array for various types of analysis. The methods are particularly useful for analyzing populations having a high degree of complexity, for example, chromosomal-derived DNA, or whole genomic DNA, or mRNA population. In addition, such methods allow for analysis of pooled samples.

### CROSS-REFERENCE TO RELATED APPLICATIONS

The present application derives priority from U.S. Ser. No. 60/228,251, filed Aug. 26, 2000, and 09/768,936 filed Jan. 23, 2001, which are incorporated by reference in their entirety for all purposes.

### BACKGROUND

The scientific literature provides considerable discussion of nucleic acid probe arrays and their use in various forms of genetic analysis (for review, see Schena, Microarray Biochip Technology (Eaton Publishing, MA, USA, 2000)). For example, nucleic acid probe arrays have been used for detecting variations in DNA sequences such as polymorphisms or species variations. Nucleic acid probe arrays have also been used for monitoring relative levels of populations of mRNA and detecting differentially expressed mRNAs.

Some methods for detecting polymorphisms using arrays of nucleic acid probes are described in WO 95/11995 (incorporated by reference in its entirety for all purposes), and a further strategy for detecting a polymorphism using an array of probes is described in EP 717,113. In this strategy, an array contains overlapping probes spanning a region of interest in a reference sequence. The array is hybridized to a labelled target sequence, which may be the same as the reference sequence or a variant thereof. Additional methods of polymorphism discovery and analysis are described in EP 0950720, which discusses use of primary arrays for de novo discovery of polymorphisms and use of secondary arrays for polymorphic profiling at the newly discovered polymorphic sites of different individuals. WO98/56954 discusses methods of identifying polymorphisms affecting expression of mRNA species.

Methods for using arrays of probes for monitoring expression of mRNA populations are described in U.S. Pat. 6,040,138, EP 853,679 and WO97/27317. Such methods employ groups of probes complementary to mRNA target sequences of interest. mRNA populations or an amplification products thereof are applied to an array, and targets of interest are identified, and optionally, quantified by determining the extent of specific binding to complementary probes. Additionally, binding of the target to probes known to be mismatched with the target can be used as a measure of background nonspecific binding and subtracted from specific binding of target to complementary probes. U.S. Ser. No. 60/203,418 and 09/853,113, incorporated by reference for all purposes, discuss methods for determining functional regions in a genome using nucleic acid probe arrays. Additional methods for transcriptional annotation are described in, for example, U.S. Ser. No. 60/206,866 filed May 24, 2000 and 09/641,081 filed Aug. 16, 2000 incorporated by reference for all purposes.

However, the clarity and quality of the results obtained when using microarrays for analysis is, to a large degree, dependent on the quality and complexity of the target nucleic acid interrogated. The present invention provides methods for improving the quality and reducing the complexity of target nucleic acids applied to arrays, thereby improving the quality of the resulting data.

### SUMMARY OF THE CLAIMED INVENTION

The invention provides several methods for reducing the complexity of a population of nucleic acids prior to performing an analysis of the population of nucleic acids on a nucleic acid probe array. Such reduction in complexity results in a subset of the initial population of nucleic acids enriched for a desired property, or lacking nucleic acids having an undesired property. The resulting nucleic acids in the subset are then applied to a nucleic acid probe array for various types of analyses. Results obtained using a sample of reduced complexity can be superior to those obtained using samples where the methods of the present invention have not been employed. In general, the signal to noise ratio for samples with less complexity is much improved over untreated samples. The methods are particularly useful for analyzing nucleic acid populations having a high degree of complexity, for example, populations of DNA spanning a chromosome, DNA spanning a whole genome, or mRNA. Further, the methods of the present invention enable pooling of target samples for analysis on an array. Pooling in appropriate circumstances leads to a reduction in cost and time of analysis if many samples must be analyzed.

Thus, the present invention provides in one aspect, a method of analyzing a subset of nucleic acids within a nucleic acid population, comprising: providing a population of nucleic acid fragments wherein at least some of said fragments have sequences that are repeated; denaturing said population of nucleic acid fragments; incubating said denatured population of nucleic acid fragments under conditions to produce a double-stranded subset of said population of nucleic acids and a single-stranded subset of said population of nucleic acids, wherein under said annealing conditions nucleic acid fragments of said population having repeat sequences preferentially anneal with each other relative to nucleic acid fragments of said population lacking repeat sequences; separating said single-stranded subset of said population of nucleic acid fragments from said double-stranded subset of said population of nucleic acid fragments; hybridizing said separated single-stranded subset of said population of nucleic acid fragments to probes on a nucleic acid probe array; and determining which of said probes on said array hybridize to said single-stranded subset of said population of nucleic acid fragments, thereby analyzing said single-stranded subset of said population of nucleic acid fragments.

In yet another aspect of the invention, there is provided a method of analyzing a subset of nucleic acids within a nucleic acid population, comprising: providing a driver population of nucleic acids and a tester population of nucleic acids; denaturing said driver population of nucleic acids and said tester population of nucleic acids; annealing said driver population to said tester population to produce a single-stranded subset of nucleic acids and a double-stranded subset of nucleic acids; immobilizing said driver population of nucleic acids to produce an unimmobilized single-stranded tester subset of nucleic acids, an immobilized double-stranded tester-driver subset of nucleic acids and an immobilized single-stranded driver subset of nucleic acids; separating said unimmobilized single-stranded tester subset of nucleic acids from said immobilized double-stranded tester-driver subset of nucleic acids and said immobilized single-stranded driver subset of nucleic acids; hybridizing said unimmobilized single-stranded tester subset of nucleic acids to probes on a nucleic acid probe array; and determining which of said probes on said array hybridize to said unimmobilized single-stranded tester subset of nucleic acids, thereby analyzing said unimmobilized singlestranded tester subset of nucleic acids.

In yet another aspect of the invention, there is provided a method of analyzing a subset of nucleic acids within a nucleic acid population, comprising: providing a driver population of nucleic acids and a tester population of nucleic acids; denaturing said driver population of nucleic acids and said tester population of nucleic acids; annealing said driver population to said tester population to produce a single-stranded subset of nucleic acids and a double-stranded subset of nucleic acids; immobilizing said driver population of nucleic acids to produce an unimmobilized single-stranded tester subset of nucleic acids, an immobilized double-stranded tester-driver subset of nucleic acids and an immobilized single-stranded driver subset of nucleic acids; separating said unimmobilized single-stranded tester subset of nucleic acids from said immobilized double-stranded tester-driver subset of nucleic acids and said immobilized single-stranded driver subset of nucleic acids; dissociating said immobilized double-stranded tester-driver subset of nucleic acids to produce a subset of complementary tester nucleic acids and a subset of immobilized complementary driver nucleic acids; separating said subset of complementary tester nucleic acids from said subset of immobilized complementary driver nucleic acids; hybridizing said subset of complementary tester nucleic acids to probes on a nucleic acid probe array; and determining which of said probes on said array hybridize to said subset of complementary tester nucleic acids, thereby analyzing said subset of complementary tester nucleic acids.

### BRIEF DESCRIPTION OF THE FIGURES

FIG. 1 shows an exemplary scheme for removing repeat sequences from a population of nucleic acid fragments. First, a population of genomic DNA is digested with a restriction enzyme or DNaseI to produce fragments of, for example, an average size of about 300 bp. The fragments are denatured and allowed to reanneal. Repeat sequences hybridize with each other, whereas nonrepeat sequences remain in single stranded form. The double-stranded hybrids and the single-stranded sequences are then separated on a hydroxyapatite HPLC column. The DNA is loaded in a phosphate buffer and eluted using a phosphate buffer gradient. Single-stranded DNA elutes at a concentration of about 120-140 mM phosphate, and double-stranded DNA elutes at a concentration of about 500 mM to 1 M phosphate. The single-stranded sequences then may be labeled prior to application to an array.

FIG. 2 shows an exemplary scheme for enriching a tester population of nucleic acids by hybridization of the tester population to a driver population of nucleic acids. In this scheme the driver DNA is a genomic clone in, for example, a BAC, YAC or PAC. The genomic clone is cleaved to fragments of average size about 300 bp using a restriction enzyme (only one strand of the double-stranded fragments is shown). The fragments are ligated to linkers containing primer sites and amplified in the presence of a biotin labeled nucleotides. The tester DNA is a cDNA population produced by reverse transcription of an MnRNA population. The cDNA is also digested with a restriction enzyme to an average length of about 300 bp, ligated with linkers containing primer sites to allow amplification, and then amplified (again, only one strand of the amplified fragments is shown). The resulting amplified cDNA fragments and biotin-labelled genomic fragments are then denatured and hybridized in solution. The genomic fragments and any hybridized cDNA are then immobilized to streptavidin labeled magnetic beads by virtue of the affinity of the streptavidin for the biotin label on the driver nucleic acids. The bead/hybrid complexes are then washed to remove unhybridized tester nucleic acids. Hybridized tester nucleic acids are then dissociated from the immobilized driver by raising the temperature or lowering the salt concentration.

FIG. 3 shows the identification of expressed sequences using the methods of the present invention. Expressed sequences were isolated from cDNA that was synthesized from a combination of 10 tissue samples, and hybridized onto Chromosome 21 genomic microarrays. The figure depicts a typical pattern of expressed sequences. The red peaks indicate expressed sequences, with previously identified exons shown as blue rectangles above the sequence peaks. The yellow bars are repeat regions that have been masked on the microarray.

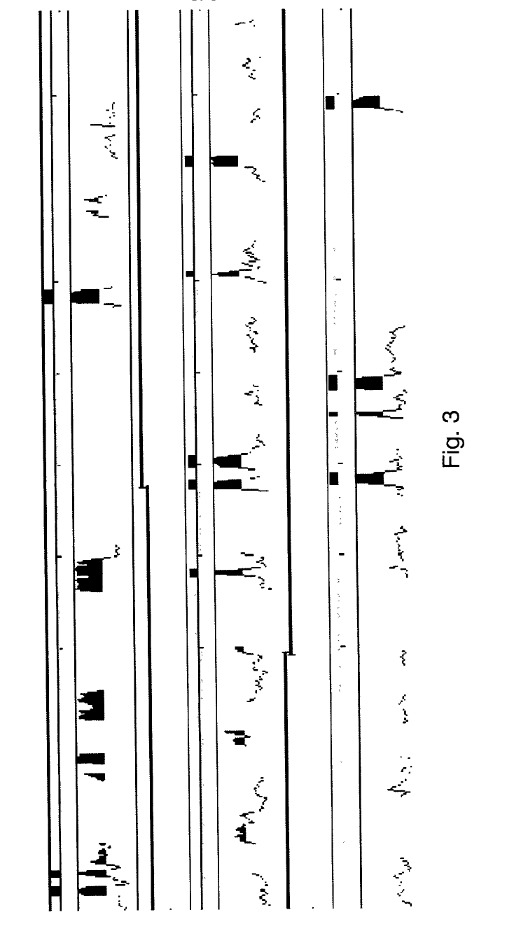

### DEFINITIONS

Unless otherwise apparent from the context, reference to mRNA populations includes nucleic acid populations derived therefrom by processes in which the mRNA serves as template for polynucleotide extension, such as cDNA or cRNA.

A nucleic acid is a deoxyribonucleotide or ribonucleotide polymer in either single- or double-stranded form, including known analogs of natural nucleotides unless otherwise indicated.

An oligonucleotide is a single-stranded nucleic acid ranging in length from 2 to about 500 bases.

A probe is a nucleic acid capable of binding to a target nucleic acid of complementary sequence through one or more types of chemical bonds, usually through complementary base pairing, usually through hydrogen bond formation. A nucleic acid probe may include natural (i.e. A, G, C, or T) or modified bases (e.g., 7-deazaguanosine, inosine). In addition, the bases in a nucleic acid probe may be joined by a linkage other than a phosphodiester bond, so long as it does not interfere with hybridization. Thus, nucleic acid probes may be peptide nucleic acids in which the constituent bases are joined by peptide bonds rather than phosphodiester linkages.

Specific hybridization refers to the binding, duplexing, or hybridizing of a molecule only to a particular nucleotide sequence under stringent conditions when that sequence is present in a complex mixture (e.g., total cellular) DNA or RNA. Stringent conditions are conditions under which a probe hybridizes to its target subsequence, but to no other sequences. Stringent conditions are sequence-dependent and are different in different circumstances. Longer sequences hybridize specifically at higher temperatures. Generally, stringent conditions are selected to be about 5° C. lower than the thermal melting point (Tm) for the specific sequence at a defined ionic strength and pH. The Tm is the temperature (under defined ionic strength, pH, and nucleic acid concentration) at which 50% of the probes complementary to the target sequence hybridize to the target sequence at equilibrium. (As the target sequences are generally present in excess, at Tm, 50% of the probes are occupied at equilibrium). Typically, stringent conditions include a salt concentration of at least about 0.01 to 1.0 M Na ion concentration (or other salts) at pH 7.0 to 8.3 and the temperature is at least about 30° C. for short probes (e.g., 10 to 50 nucleotides). Stringent conditions can also be achieved with the addition of destabilizing agents such as formamide. For example, conditions of 5×SSPE (750 mM NaCl, 50 mM NaPhosphate, 5 mM EDTA, pH 7.4) and a temperature of 25-30° C. are suitable for allele-specific probe hybridizations.

A perfectly matched probe has a sequence perfectly complementary to a particular target sequence. The test probe is typically perfectly complementary to a portion (subsequence) of the target sequence. The term “mismatch probe” refers to probes whose sequence is deliberately selected not to be perfectly complementary to a particular target sequence. Although the mismatch(es) may be located anywhere in the mismatch probe, terminal mismatches are less desirable as a terminal mismatch is less likely to prevent hybridization of the target sequence. Thus, probes are often designed to have the mismatch located at or near the center of the probe such that the mismatch is most likely to destabilize the duplex with the target sequence under the test hybridization conditions.

A polymorphic marker or site is the locus at which divergence occurs. Preferred markers have at least two alleles, each occurring at frequency of greater than 1%, and more preferably greater than 10% or 20% of a selected population. A polymorphic locus may be as small as one base pair. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The first identified allelic form is arbitrarily designated as the reference form and other allelic forms are designated as alternative or variant alleles. The allelic form occurring most frequently in a selected population is sometimes referred to as the wildtype form. Diploid organisms may be homozygous or heterozygous for allelic forms. A diallelic polymorphism has two forms. A triallelic polymorphism has three forms. A single nucleotide polymorphism (SNP) occurs at a polymorphic site occupied by a single nucleotide, which is the site of variation between allelic sequences. The site is usually preceded by and followed by highly conserved sequences of the allele (e.g., sequences that vary in less than 1/100 or 1/1000 members of the populations). A single nucleotide polymorphism usually arises due to substitution of one nucleotide for another at the polymorphic site. Single nucleotide polymorphisms can also arise from a deletion of a nucleotide or an insertion of a nucleotide relative to a reference allele.

### DETAILED DESCRIPTION

The present invention provides several methods for reducing the complexity of a population of nucleic acids prior to performing an analysis of the nucleic acids on a nucleic acid probe array. The results obtained using nucleic acid array technologies are enhanced by reducing complexity of the target or sample nucleic acids applied to the array. The methods result in a subset of the initial population enriched for a desired property, or lacking nucleic acids having an undesired property, and the resulting nucleic acids in the subset are then applied to the array for various types of analyses. The methods are particularly useful using nucleic acid probe arrays to analyze nucleic acid populations having a high degree of complexity, for example, populations of chromosomal DNA, or whole genomic DNA, or mRNA. The methods of the present invention attain reduced complexity of samples which enables analysis of pooled samples.

In some methods, an initial population of nucleic acids is treated so as to reduce or eliminate fragments having repeat sequences. In general, nonrepeat sequences contain the coding and key regulatory regions of genomic DNA and are of interest for most subsequent genetic analyses. Repeat sequences can be eliminated by a process that involves denaturing the initial population (if double-stranded), and reannealing. Single stranded nucleic acids with repeat sequences preferentially hybridize with each other relative to single stranded nucleic acids of unique sequence because there is a greater probability of nucleic acids with repeated regions finding a complementary nucleic acid with which to hybridize (see, e.g., Ryffel et al., 1975, Experientia (BASEL) 31 (6) 746; Ryffel et al., 1975, Biochemistry 14(7) 1385-1389; Ryffel et al., Biochemistry 14(7) 1379-1385; Marsh et al., 1973, Biochem. Biophys. Res. Comm. 55(3) 805-811; Krueger and McCarthy, 1970, Fed. Proc. 29 (2) 757; Tereba and McCarthy, 1973, Biochem. 12(23) 4675-4679, all incorporated in their entities by reference for all purposes). After annealing, double-stranded (annealed) and single-stranded nucleic acids are separated from one another, and the resulting single-stranded nucleic acids are thus enriched for nonrepeat sequences. These single-stranded, enriched sequences are then applied to a nucleic acid probe array for a variety of genetic analyses. For example, such analyses include de novo polymorphic site discovery, detection of a plurality of predetermined polymorphic sites, SNP analysis, expression analysis and the like. In general, when analyzing arrays, it is desirable to discriminate between specific hybridization between complementary sequences and nonspecific hybridization between probes and target sequences. Reducing the complexity of the target nucleic acid leads to reduction in non-specific hybridization, resulting in less non-specific “noise” and a more robust hybridization signal—particularly important when analyzing target samples that may have low copy numbers of some sequences.

Thus, the present invention provides in one aspect, a method of analyzing a subset of nucleic acids within a nucleic acid population, comprising: providing a population of nucleic acid fragments wherein at least some of the fragments have sequences that are repeated; denaturing the population of nucleic acid fragments; incubating the denatured population of nucleic acid fragments under conditions to produce a double-stranded subset of the population of nucleic acids and a single-stranded subset of the population of nucleic acids, wherein under the annealing conditions nucleic acid fragments of the population having repeat sequences preferentially anneal with each other relative to nucleic acid fragments of the population lacking repeat sequences; separating the single-stranded subset of the population of nucleic acid fragments from the double-stranded subset of the population of nucleic acid fragments; hybridizing the separated single-stranded subset of the population of nucleic acid fragments to probes on a nucleic acid probe array; and determining which of the probes on the array hybridize to the single-stranded subset of the population of nucleic acid fragments, thereby analyzing the single-stranded subset of the population of nucleic acid fragments. In some embodiments of this aspect of the invention, the population of nucleic acid fragments are genomic DNA fragments, and may be from a human genome. In some specific embodiments, the fragments from the human genome are fragments from the same chromosome of different individuals. Also, in this aspect of the present invention, the separating step may be performed by column chromatography, and in specific embodiments, the column used is a hydroxyapatite column. Further, the separating step is performed under conditions whereby the single-stranded subset and the double-stranded are eluted in phosphate buffer. In an alternative embodiment, the separating step is performed by HPLC, and in yet another embodiment, the separating step is performed by successively performing hydroxyapatite chromatography and HPLC. Also in this aspect of the invention, the probe array may comprise a set of probes complementary to a known reference sequence, where the reference sequence is substantially identical to the sequence of the population of nucleic acid fragments. For example, in this aspect of the invention, the population of nucleic acid fragments may be from a chromosome from a first individual, and the reference sequences may be from a corresponding chromosome from a second individual. Alternatively, the population of nucleic acid fragments may be genomic fragments from a first individual, and the reference sequences may be genomic fragments from a second individual of a species closely related to the first individual. For example, the population of nucleic acid fragments may be genomic fragments from a non-human primate, and the reference sequence may be from a human. In yet another example, the population of nucleic acid fragments may be genomic fragments from a non-human mammal, and the reference sequence may be from a human.

In yet another aspect of the invention, there is provided a method of analyzing a subset of nucleic acids within a nucleic acid population, comprising: providing a driver population of nucleic acids and a tester population of nucleic acids; denaturing the driver population of nucleic acids and the tester population of nucleic acids; annealing the driver population to the tester population to produce a single-stranded subset of nucleic acids and a double-stranded subset of nucleic acids; immobilizing the driver population of nucleic acids to produce an unimmobilized single-stranded tester subset of nucleic acids, an immobilized double-stranded tester-driver subset of nucleic acids and an immobilized single-stranded driver subset of nucleic acids; separating said unimmobilized single-stranded tester subset of nucleic acids from the immobilized double-stranded tester-driver subset of nucleic acids and the immobilized single-stranded driver subset of nucleic acids; hybridizing the unimmobilized single-stranded tester subset of nucleic acids to probes on a nucleic acid probe array; and determining which of the probes on the array hybridize to the unimmobilized single-stranded tester subset of nucleic acids, thereby analyzing the unimmobilized single-stranded tester subset of nucleic acids. In one embodiment of this aspect of the invention, driver population of nucleic acids may each bear a tag by which the driver population of nucleic acids can be immobilized to a binding moiety with affinity for the tag. For example, the tag may be biotin, and the binding moiety may be avidin or streptavidin. In certain embodiments, the separating step is performed by immobilizing the immobilized double-stranded tester-driver subset of nucleic acids and the immobilized single-stranded driver subset of nucleic acids via the tags on the driver population. Additionally, in some embodiments, the driver population of nucleic acids are genomic DNA from a first source, and the tester population of nucleic acids are genomic DNA from a second source. For example, the first source may be mRNA from a tissue of a first species, and the second source may be mRNA from the same tissue of a different species. Alternatively, for example, the first source may be from a first tissue of a first species, and the second source may be from a different tissue of the first species. In some embodiments of this aspect of the invention, the immobilizing step is performed before the annealing step, or the immobilizing step may be performed before the denaturing step.

In yet another aspect of the invention, there is provided a method of analyzing a subset of nucleic acids within a nucleic acid population, comprising: providing a driver population of nucleic acids and a tester population of nucleic acids; denaturing the driver population of nucleic acids and the tester population of nucleic acids; annealing the driver population to the tester population to produce a single-stranded subset of nucleic acids and a double-stranded subset of nucleic acids; immobilizing the driver population of nucleic acids to produce an unimmobilized single-stranded tester subset of nucleic acids, an immobilized double-stranded tester-driver subset of nucleic acids and an immobilized single-stranded driver subset of nucleic acids; separating the unimmobilized single-stranded tester subset of nucleic acids from the immobilized double-stranded tester-driver subset of nucleic acids and the immobilized single-stranded driver subset of nucleic acids; dissociating the immobilized double-stranded tester-driver subset of nucleic acids to produce a subset of complementary tester nucleic acids and a subset of immobilized complementary driver nucleic acids; separating the subset of complementary tester nucleic acids from the subset of immobilized complementary driver nucleic acids; hybridizing the subset of complementary tester nucleic acids to probes on a nucleic acid probe array; and determining which of the probes on the array hybridize to the subset of complementary tester nucleic acids, thereby analyzing the subset of complementary tester nucleic acids. In one embodiment of this aspect of the invention, driver population of nucleic acids may each bear a tag by which the driver population of nucleic acids can be immobilized to a binding moiety with affinity for the tag. For example, the tag may be biotin, and the binding moiety may be avidin or streptavidin. In certain embodiments, the separating step is performed by immobilizing the immobilized double-stranded tester-driver subset of nucleic acids and the immobilized single-stranded driver subset of nucleic acids via the tags on the driver population. Additionally, in some embodiments, the driver population of nucleic acids are genomic DNA from a first source, and the tester population of nucleic acids are genomic DNA from a second source. For example, the first source may be from a tissue of a first species, and the second source may be from the same tissue of a different species. Alternatively, for example, the first source may be MRNA from a first tissue of a first species, and the second source may be mRNA from a different tissue of the first species. In alternative embodiments, driver population or the tester population or both the driver and the tester populations is a PCR amplification product. In another embodiment, the driver population is from a plurality of noncontiguous regions of a genome of a species, and in certain embodiments, the driver population is from at least ten noncontiguous regions. In addition, the driver population may be MRNA or nucleic acids derived therefrom, and the tester population may be genomic DNA. In another embodiment, the driver population may be mRNA or nucleic acids derived therefrom from a first source, and the tester population may be mRNA or nucleic acids derived therefrom from a second source. In some embodiments of this aspect of the invention, the immobilizing step is performed before the annealing step, or the immobilizing step may be performed before the denaturing step.

Repeat sequences are sequences occurring occur more than once in a haploid genome of a single organism. In some instances, multiple copies of a repeat sequence are identical. In other instances, there are some divergences between copies but substantial sequence identity, e.g., at least 80 or 90%. More than 30% of human DNA consists of sequences repeated at least 20 times. Families of repeated DNA sequences of 100-500 bp that are interspersed throughout the genome are sometimes known as SINES (short interspersed repeats). Alu sequences are examples of SINES that are about 300 bp and occur almost 1 million times in the human genome. Longer interspersed repeat sequences of 1 kb or more are known as LINES (long interspersed repeats). Some repeat sequences are not interspersed throughout the genome but are concentrated at particular loci. These repeats are known as satellite repeats. Some repeat sequences are actual genes, such as the genes that code for ribosomal RNAs and histones. However, the function, if any, of most repeat sequences is unclear. The vast majority of protein coding sequences and their associated regulatory sequences occur in single copy regions of the genome.

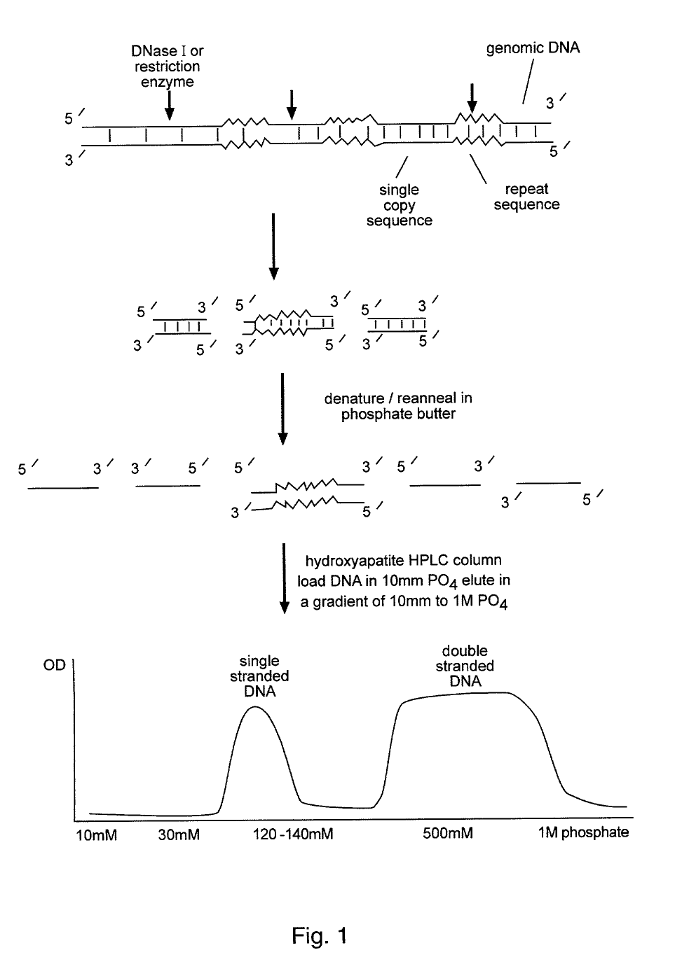

One aspect of the present invention provides methods for enriching for single copy regions of a genome relative to repeat sequences before performing a genetic analysis using a nucleic acid probe array (see FIG. 1). The starting population of fragments for enrichment can be from a whole genome, a collection of chromosomes, a single chromosome, or one or more regions from one or more chromosomes. In some methods, the fragments are overlapping fragments spanning a length of 100 kb, 1 Mb, 10 Mb or 100 Mb. The fragments may be obtained from the same individual, which can be a human or other mammal or other species.

Genomic fragments may be produced by fragmenting an initial substrate such as an isolated chromosome or genome. Also, the initial substrate can be amplified, and/or labelled before or after fragmentation. Both enzymatic and mechanical methods can be used for fragmentation. The fragmenting can be effected by restriction digestion, often using a partial digest with a restriction enzyme with a short recognition site or a limited digest with a mixture of enzymes or with DNaseI. Alternatively, fragments can be produced by sonication, or by PCR amplification using random primers or random fragments of an initial substrate. Other suitable methods include mechanic or liquid shearing by using a French press or a UCHGR Shearing Device. In some methods, fragments are attached to linkers at one or both ends to provide primer sites for subsequent amplification. In some methods, fragments have an average size of about 300 bp. For example, appropriate restriction enzymes may be used to cut genomic DNAs to a desired range of sizes. Fragments containing repeat sequences are removed from the population by a combination of denaturation (assuming the fragments are double stranded) and reannealing. Denaturation can be effected by heating fragments in excess of the average melting point of the fragments. The denatured fragments are then cooled to below the average melting point (e.g., about 25 degrees below the average melting point) for reannealing. The reassociation can be followed by, for example, monitoring hyperchromicity at 260 nm. As DNA renatures, the hyperchromicity increases due to greater absorbance of double stranded relative to single stranded DNA. The hyperchromicity curve shows a point of inflexion at which half of the DNA is reannealed. The reannealing reaction is often stopped about this time, but the duration of the reaction can be adjusted depending on the percentage of repetitive DNA in the sample. The more repetitive DNA sequences, the longer the annealing reaction should proceed. The reannealing reaction can effectively be stopped by rapid cooling of the annealing mixture to just above freezing.

After the annealing reaction, annealed double-stranded DNA is separated from single-stranded DNA. Separation can be effected using column chromatography. A hydroxyapatite (calcium phosphate) column is particularly suitable (see Ryffel & McCarthy, Biochemistry, 14.7, 1385-1389 (1975) incorporated by reference for all purposes). Both single- and double-stranded nucleic acids bind to the column at low phosphate concentration (10-30 mM sodium phosphate). At intermediate phosphate concentrations (120 mM to 140 mM,), single-stranded DNA no longer binds the column, however, double-stranded DNA continues to bind. At higher concentrations (400 mM), both single- and double-stranded DNA no longer bind to the column. Thus, DNA can be loaded on the column at low phosphate concentration, in which case both single- and double-stranded nucleic acids bind. Single-stranded nucleic acids are then eluted with an increasing concentration gradient of sodium phosphate buffer. Alternatively, single- and double-stranded nucleic acids can be loaded at an intermediate phosphate concentration, in which case the single-stranded nucleic acids pass though without binding and the double-stranded nucleic acids binds (see Genome Analysis: A Laboratory Manual, Volume 2, Detecting Genes (eds. Bruce Birren et al., Cold Spring Harbor Press, 1998)). In some methods, hydroxyapatite columns are combined with HPLC. Alternatively, or additionally, the annealing reaction mixture can be treated with a nuclease that selectively digests double-stranded DNA.

After separation of single-stranded nucleic acids from double-stranded nucleic acids, the single-stranded nucleic acids can be applied directly to an array, or can be the subject of additional treatment (for example, labeling reactions or amplification reactions) before application to an array. For example, in some methods, the single-stranded fragments are allowed to anneal with each other, forming double-stranded fragments, which are then amplified, labelled, and denatured before being applied to the array. In some methods, single-stranded nucleic acids that were not previously labeled are now labelled before application to an array. Some methods for end-labelling fragments are described by WO97/27317. In some methods, the single-stranded fragments are broken down to still smaller fragments before being applied to an array.

The type of array to which the fragments are applied of course depends on the form of contemplated analysis. In some methods, fragments are applied to arrays designed for de novo polymorphism discovery. These arrays typically contain overlapping probes tiling a region of a known reference sequence. The hybridization pattern of the fragments to the array indicates the site and nature of points of divergence between the sequence of the fragments and the reference sequence, and hence the location and identity of polymorphic sites. In other methods, the fragments are applied to an array designed to detect a collection of polymorphisms where the location and nature of polymorphic forms is already known. In such methods, the hybridization pattern of the nucleic acid fragments to the array indicates a polymorphic profile of the individual from whom the fragments were obtained (i.e., a matrix of polymorphic sites, and polymorphic forms present in those sites).

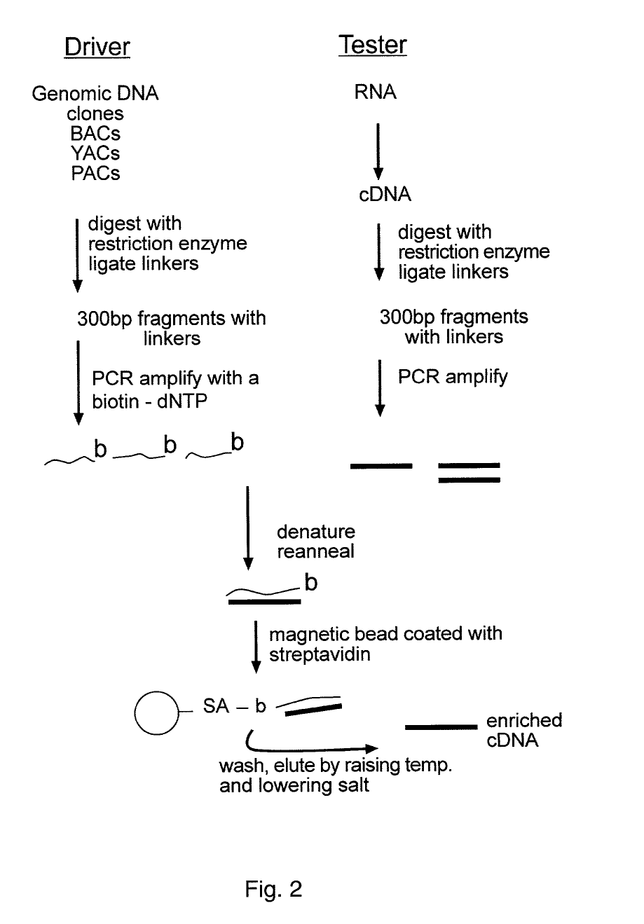

A variety of enrichments can be performed by hybridization of tester nucleic acids to driver nucleic acids as described herein (for example, see FIG. 2). In these methods, either or both driver and tester nucleic acids can be amplified before the enrichment procedure. In one embodiment, driver and/or tester nucleic acids are fragmented before performing the hybridization reaction. Fragmentation can be achieved by any of the methods described above, usually to an average size of about 200-700 bp or about 250-500 bp. Fragmentation before enrichment is typical with genomic populations and possible, but not usual, with MRNA populations. In some embodiments, a population of nucleic acids is fragmented, the fragments are ligated to oligonucleotides having primer sites, and the ligated fragments are amplified. Also, the tester nucleic acid fragments can be labelled. Labelling can be performed before or after the enrichment procedure. In these methods, populations of driver and tester nucleic acid fragments are denatured (if initially double stranded), mixed (if denaturation was performed separately for each population) and allowed to reanneal.

As in the methods for eliminating repeat sequences, denaturation can be performed by raising the temperature over the average melting point of driver and tester nucleic acid populations. The two populations can be denatured separately or together. Hybrids between tester and driver nucleic acids are separated from unhybridized tester nucleic acid. Separation can be effected by inclusion of a tag on all driver fragments and immobilizing the driver fragments to a binding moiety. For example, a biotin tag can be attached to driver fragments by amplifying them using a biotin labelled primer or biotin labelled nucleotides or by ligating them to biotin labeled oligonucleotides or by directly attaching biotin to the fragments (see e.g., Birren et al. supra, at ch. 3). Biotin labelled driver fragments can then be immobilized to a support bearing an avidin or streptavidin binding moiety. For example, magnetic beads coated with streptavidin, available from Dynal (Norway), are suitable for immobilizing biotin-labelled DNA. Procedures for performing enrichments of cDNA using immobilized DNA on beads are described by Birren et al., supra at ch. 3. Other combinations of tag and binding moiety similarly can be used. Alternatively, hybrids can be separated from single-stranded fragments using hydroxyapatite chromatography as described above. Alternatively, separation can be effected using a nuclease that digests duplex nucleic acids without digesting single stranded nucleic acids or vice versa. For example, S1 nuclease preferentially digests single stranded DNA, whereas most restriction enzymes preferentially digest double stranded DNA.

In some methods, the driver population is genomic DNA and the tester population is an mRNA population or nucleic acid population derived therefrom (e.g., cDNA or cRNA). As will become apparent, such methods serve to normalize the representation of different nucleic acid sequence species within the mRNA population (or nucleic acids derived therefrom). In other words, the methods enrich the representation of rare mRNA species relative to the more common mRNA species. In such methods, the driver population can be from a whole genome, a chromosome, a collection of chromosomes or one or more regions of one or more chromosomes. If an entire genome is included, then the enriched population of mRNAs includes mRNAs spread throughout the genome. If a single chromosome is included, then the enriched population of mRNAs is restricted to mRNAs hybridizing to that chromosome, and so forth. The mRNA population used as the tester population can be from a single tissue type, from a cell line or from a mixture of tissue types. If from a single tissue type, the mRNA population and the resulting enriched population contains a bias toward the mRNAs expressed in that cell type. If the mRNA population is from a representative mixture of tissue types, then the population and the subsequent enriched populations contains most or substantially all (e.g., at least 50% , 75% or 90%) of mRNAs expressed by the organism. Some cell lines, such as HeLa cells, also express a substantial proportion of all mRNAs typically expressed in an organism. If cDNA or cRNA is prepared from mRNA, the preparation can be performed under conditions that preserve the relative representations of mRNA species in the original population as described by U.S. Ser. No. 6,040,138. However, such is generally not necessary because the proportions are, of course, deliberately changed in the enrichment procedure. Thus, conventional methods of cDNA preparation using polyT primers or random hexamers can be used (see Birren et al., supra at ch. 3). In some methods, adapters are ligated to cDNA to facilitate subsequent amplification or labelling.

When driver genomic DNA is hybridized with tester mRNA (or a nucleic acid derived therefrom), the mRNA hybridizes to complementary sequences in the genomic DNA sequences. However, in general, each mRNA species has only a single complementary genomic DNA sequence in a haploid genome. Accordingly, highly represented mRNA species and minimally represented species (and intermediately represented sequences) in general all hybridize to genomic DNA to a similar extent. In theory, one molecule of mRNA should hybridize per haploid genome for a single copy gene. In practice, this ratio is not observed for all single copy genes due to the presence of introns. For example, a gene having ten spaced exons can hybridize to different regions of ten copies of the same mRNA. Nevertheless, the hybridization does result in substantial normalization between mRNA species. For example, whereas the variation copy number between species in an unnormalized population can be greater than 10⁵, in a normalized population, the variation is more typically within a factor of 1000, 100, or 10.

After performing hybridization, hybrids between tester and driver populations are separated from unhybridized tester. The unhybridized tester is set aside. Tester nucleic acids complementary to driver nucleic acids are then dissociated from the complementary driver nucleic acids (e.g., by raising the temperature above the melting point). The driver nucleic acids remain associated with the solid phase, and the resulting subset of complementary tester nucleic acids are obtained in solution. The resulting subset of complementary tester nucleic acids are initially in single-stranded form. The single stranded fragments can be labelled (if not labelled already) and applied directly to an array. Alternatively, the fragments can be renatured with each other, for amplification and labeling. Amplified fragments are then denatured again before being applied to an array.

The subset of tester fragments obtained can be subject to a variety of genetic analyses. In some methods, the fragments are used for de novo polymorphism discovery, in similar fashion to that described above. The polymorphisms discovered thereby are highly likely to occur within expressed regions of the genome. The subset of tester fragments can also be used for polymorphic profiling of previously characterized polymorphic sites within expressed regions within an individual. Use of mRNA populations has advantages relative to use of genomic DNA in that nonexpressed regions of the genome, which probably contain relatively few polymorphic sites of functional significance but which would otherwise contribute to a background of nonspecific binding on the array, are not applied to the array. It is estimated that only 5% of the human genome contains coding regions.

The subset of tester fragments can also be used for discovering relatively rare differentially expressed genes. For example, by comparing tester populations, enriched as described above from different tissue types, one can identify species within one tester population that are not expressed within another. Such MRNA species can be cloned as described in WO97/27317. This type of analysis is particularly useful for identifying genes that are expressed at a low level in one tissue, and not at all in another tissue.

In some methods, both driver and tester populations are genomic but from different sources. In some methods, the different sources are different individuals from the same species, in others, the different sources are individuals from different species. For example, the two sources can be two different humans, or one human and one cat, or one mouse and one dog, and so forth. Such methods serve to enrich either fragments that are common to the two sources or fragments that differ between the two sources. For the former type of enrichment, one retains tester fragments hybridizing to driver fragments. For the latter type of enrichment, one retains tester fragments not hybridizing to driver fragments. Common sequences are of interest because commonality often implies evolutionary conservation; hence, a possible important functional role. Polymorphisms occurring within regions that are conserved between species are more likely to have phenotypic consequences. Accordingly, given the vast number of polymorphic sites within a genome, it can be advantageous to focus on conserved regions for polymorphism discovery, and/or to use polymorphisms within conserved regions for association studies. Disparate sequences between sources are also of interest, because these sequences are the locus of genetic diversity between different individuals and/or species.

In these, as in other methods, driver and tester populations can be obtained from whole genomes, collections of chromosomes, individual chromosomes or one or more regions of individual chromosomes. Usually, the fragments within a driver population are obtained from the same individual, as is the case for the fragments within a tester population; however, the driver and tester populations are generally obtained from different individuals. Either driver and/or tester populations can be amplified before performing hybridization. The tester population can be labelled before or after the hybridization. If the goal is to isolate sequences that are common between the driver and tester populations, the nonhybridizing subset of nucleic acids from the tester population are set aside, and the subset of tester fragments hybridizing to the driver are dissociated from the driver. These fragments can be subject to amplification and/or labelling before being applied to an array. If the goal is to isolate disparate fragments between the driver and tester populations, then the driver and tester fragments that hybridize are set aside and the nonhybridizing tester fragments are applied to an array (optionally with labelling, if not already labelled). Alternatively, the nonhybridizing tester fragments can be hybridized with each other, amplified and labeled before being applied to an array.

In other methods, hybridization between driver and tester fragments is used as a surrogate for selective amplification of a certain region of genomic DNA. The goal in such methods is to apply one or more regions of genomic DNA to an array without applying others. Such could be achieved by selective amplification of the desired regions. However, performing selective amplification on a large number of samples, particularly if the amplification is a multiplex amplification of multiple noncontiguous regions, can be tedious and subject to error. Alternatively, the amplification can be performed on a single genomic sample, and the amplified sample then used as a driver population to enrich equivalent regions from a broader initial population of tester DNA. For example, the driver population can be a long range PCR product of a particular chromosome, or a YAC or BAC clone within a particular chromosome. The tester population can be a whole genomic population or the whole chromosome from which the BAC, YAC or long range PCR product was obtained. When the tester population is annealed with the driver population, substantially only the complementary fragments within the tester population hybridize. These fragments can then be dissociated from the driver and applied to an array (optionally with labelling, if not already labelled). The fragments can be used for de novo polymorphism discovery or polymorphic profiling as described in other methods. The benefits of such enrichment are particularly evident when it desired to analyze a plurality of noncontiguous regions within a genome (e.g., ten or more), and/or when it desired to analyze tester DNA from a plurality of individuals (e.g., ten or more).

In other methods, a driver population of mRNA or nucleic acids derived therefrom is used to enrich a tester population of genomic DNA. Such methods enrich the genomic DNA population for fragments represented in the mRNA. The enrichment results in a population of nucleic acids that are normalized in copy number relative to the original population of mRNA. In addition, the enriched nucleic acids include regions of genomic DNA proximate to expressed regions, such as intron-exon borders, and nonexpressed regulatory sequences, such as promoters and enhancers. The enriched population can be used in similar analyses to those described above. In addition, the population is useful for discovering and detecting polymorphisms in nonexpressed regions of DNA that cannot be detected by analysis of mRNA populations. Such polymorphisms can have roles in regulating the extent of expression of a gene.

The tester population can be from a whole genome, a chromosome, a collection of chromosomes or one or more regions of one or more chromosomes. If an entire genome is included, then the enriched population of nucleic acids typically includes nucleic acids spread throughout the genome. If a single chromosome is included, then the enriched population of nucleic acids is of course within this chromosome. The mRNA population used as the driver population can be from a single tissue type, from a cell line or from a mixture of tissue types, also as described above. After hybridization of driver and tester populations, unhybridized tester fragments are set aside. Hybridized tester fragments are dissociated from the driver fragments. The resulting tester fragments can the be applied to an array (optionally with labelling, if not already labelled). Alternatively, the resulting tester fragments can be renatured, amplified, and optionally, labelled before being applied to an array.

In some methods, both driver and tester populations are mRNA populations from different sources. The different sources can be different tissues from an individual or individuals within the same species. Alternatively, the different sources can be the same tissue type from different species, (e.g., human and mouse, cat, dog, horse, cow, sheep, primate and so forth). In a further variation, the two sources can be the same tissue subject to different environmental factors, for example, exposure to a drug or potentially toxic compound. The enrichment can be used to enrich either for fragments that are common to the two populations or for fragments that are differentially represented between the two populations. Fragments that are common to the two populations of mRNA from the different sources are enriched for sequences that have been subject to evolutionary conservation. As previously discussed, polymorphisms within such sequences are particularly likely to have phenotypic consequences. Accordingly, such common fragments are useful for de novo polymorphism discovery and profiling of previously characterized polymorphisms. Differentially expressed mRNA species can also be used for polymorphism analysis, or be applied to expression monitoring arrays for identification and further characterization of the genes encoding such mRNA species. For example, such mRNA species can be applied to probe arrays containing large numbers of random probes. Probes showing specific hybridization can then be used as primers or probes to isolate genes responsible for differentially expressed mRNAs. Alternatively, the mRNA species can be hybridized to an expression monitoring array containing probes for known mRNA species. If the mixture of differentially expressed mRNAs resulting from enrichment is one of the known mRNA species, this is indicated by the resulting hybridization pattern.

As in other methods, common mRNA species between the two populations are isolated by separating the nonhybridizing tester mRNA fragments from the hybridizing double-stranded fragments, dissociating the double-stranded fragments and separating the tester mRNA from driver mRNA. In addition, the dissociated tester mRNA can be subjected to amplification and labelling before applying to an array. Amplification, if any, can be conducted with or without preservation of relative copy number of amplified species.

As previously discussed, a variety of probe array designs can be used in the invention depending on the intended type of genetic analysis. Probe arrays and their uses are reviewed in Schena, Microarray Biochip Technology (Eaton Publishing, MA, USA, 2000). Some arrays are designed for de novo discovery of polymorphisms. Such arrays contain at least a first set of probes that tiles one or more reference sequences (or regions of interest therein), and the reference sequence can be a chromosome, a genome, or any part thereof. Tiling means that the probe set contains overlapping probes, which are complementary to and span a region of interest in the reference sequence. For example, a probe set might contain a ladder of probes, each of which differs from its predecessor in the omission of a 5′ base and the acquisition of an additional 3′ base. The probes in a probe set may or may not be the same length. Such arrays typically contain at least one probe for each base to be analyzed.

Arrays for de novo polymorphism detection are hybridized to target nucleic acid samples prepared by one of the enrichment methods described above and/or to a control sample known to contain the reference sequence(s) tiled by the array. Alternatively, such an array can be hybridized simultaneously to more than one target sample or to a target sample and reference sequence by use of two-color labelling (e.g., the reference sequence bears one label and a target sample bears a second label). If the array is hybridized to a control reference sequence (or a target sequence that is identical to the reference sequence), all probes in the first probe set specifically hybridize to the reference sequence. If the array is hybridized to a target sample containing a target sequence that differs from the reference sequence at a polymorphic site, then probes flanking the polymorphic site do not show specific hybridization, whereas other probes in the first probe set distal to the polymorphic site do show specific hybridization. The existence of a polymorphism is also manifested by differences in normalized hybridization intensities of probes flanking the polymorphism relative to the probes when hybridized to corresponding targets from different individuals. For example, relative loss of hybridization intensity in a “footprint” of probes flanking a polymorphism signals a difference between the target and reference (i.e., a polymorphism) (see EP 717,113, incorporated by reference in its entirety for all purposes). Additionally, hybridization intensities for corresponding targets from different individuals can be classified into groups or clusters suggested by the data, not defined a priori, such that isolates in a given cluster tend to be similar and isolates in different clusters tend to be dissimilar. See WO 97/29212 (incorporated by reference in its entirety for all purposes).

Primary arrays of probes can also contain second, third and fourth probe sets as described in WO 95/11995. The probes from the three additional probe sets are identical to a corresponding probe from the first probe set except at the interrogation position, which occurs in the same position in each of the four corresponding probes from the four probe sets, and is occupied by a different nucleotide in the four probe sets. After hybridization of such an array to a labelled target sequence, analysis of the pattern of label should reveal the nature and position of differences between the target and reference sequence. For example, comparison of the intensities of four corresponding probes reveals the identity of a corresponding nucleotide in the target sequences aligned with the interrogation position of the probes. The corresponding nucleotide is the complement of the nucleotide occupying the interrogation position of the probe showing the highest intensity.

Additionally, arrays for de novo polymorphism detection can tile both strands of reference sequences. Both strands are tiled separately using the same principles described above, and the hybridization patterns of the two tilings are analyzed separately. Typically, the hybridization patterns of the two strands indicate the same results (i.e., location and/or nature of polymorphic form) increasing confidence in the analysis. Occasionally, there may be an apparent inconsistency between the hybridization patterns of the two strands due to, for example, base-composition effects on hybridization intensities. Such inconsistency signals the desirability of rechecking a target sample either by the same means or by some other sequencing methods, such as use of an ABI sequencer.

Arrays used for analyzing previously identified polymorphisms typically differ from the arrays for de novo identification in the following respects. First, whereas probes are typically included to span the entire length of a reference sequence in de novo discovery arrays, in arrays for analyzing precharacterized polymorphisms only a segment of a reference sequence containing a polymorphic site and immediately flanking bases typically is spanned. For example, this segment is often of a length commensurate with that of the probes. Second, an array for analyzing precharacterized polymorphisms typically includes at least two groups of probes. The first group of probes is designed based on the reference sequence, and the second group is designed based on a polymorphic form thereof. If there are three polymorphic forms at a given polymorphic site, a third group of probes can be included. Finally, because fewer probes are generally required to analyze precharacterized polymorphisms than in the de novo identification of polymorphisms, the former arrays often are designed to detect more different polymorphic sites than primary arrays. For example, whereas a de novo polymorphism discovery array may tile a single chromosome, an array for analyzing precharacterized polymorphisms can easily analyze 1,000, 10,000, 100,000 or 1,000,000 polymorphic sites in reference sequences dispersed throughout the human genome.

The design of suitable probe arrays for analysis of predetermined polymorphisms and interpretation of the hybridization patterns is described in detail in WO 95/11995; EP 717,113; and WO 97/29212. Such arrays typically contain first and second groups of probes, which are designed to be complementary to different allelic forms of the polymorphism. Each group contains a first set of probes, which is subdivided into subsets, one subset for each polymorphism. Each subset contains probes that span a polymorphism and proximate bases and are complementary to one allelic form of the polymorphism. Thus, within the first and second probe groups there are corresponding subsets of probes for each polymorphism. The hybridization patterns of these probes to target samples can be analyzed by footprinting or cluster analysis, as described above. For example, if the first and second probe groups contain subsets of probes respectively complementary to first and second allelic forms of a polymorphic site spanned by the probes, then on hybridization of the array to a sample that is homozygous for the first allelic form, all probes in the subset from the first group show specific hybridization, whereas probes in the subset from the second group that span the polymorphism show only mismatch hybridization. The mismatch hybridization is manifested as a footprint of probe intensities in a plot of normalized probe intensity (i.e., target/reference intensity ratio) for the subset of probes in the second group. Conversely, if the target sample is homozygous for the second allelic form, a footprint is observed in the normalized hybridization intensities of probes in the subset from the first probe group. If the target sample is heterozygous for both allelic forms, then a footprint is seen in normalized probe intensities from subsets in both probe groups although the depression of intensity ratio within the footprint is less marked than in footprints observed with homozygous alleles.

Alternatively, the first and second groups of probes can contain first, second, third and fourth probe sets. Each of the probe sets can be subdivided into subsets, one for each polymorphism to be analyzed by the array. The first set of probes in each group spans a polymorphic site and proximate bases and is complementary to one allelic form of the site. The second, third and fourth sets, each have a corresponding probe for each probe in the first probe set, which is identical to a corresponding probe from the first probe set except at the interrogation position, which occurs in the same position in each of the four corresponding probes from the four probe sets and is occupied by a different nucleotide in the four probe sets.

Arrays for analyzing precharacterized polymorphisms are interpreted in similar manner to the arrays for polymorphism discovery having four sets of probes described above. For example, consider an array having first and second groups of probes, where each group has four sets of probes based on first and second allelic forms of a single polymorphic site. This array is then hybridized to a target containing a homozygous first allele. The probes from the first probe set of the first group all show perfect hybridization to the target sample, and probes from the other probe sets in the first group all show mismatch hybridization. All probes from the second group of probes show at least one mismatch except the one of the four corresponding probes having an interrogation position aligned with the polymorphic site and having the same sequence as the first probe set of the first group that hybridized to the target. A probe from the second, third or fourth probe set having an interrogation position occupied by a base that is the complement of the corresponding base in the first allelic form shows specific hybridization.

If such an array is hybridized to a target sample containing homozygous second allelic form, the mirror image hybridization pattern is observed. That is, all probes in the first probe set of the second group show matched hybridization, and probes from the second, third and fourth probe sets in the second probe group show mismatch hybridization. All but one probe in the first group of probes shows mismatch hybridization. The one probe showing perfect hybridization has an interrogation site aligned with the polymorphic site and occupied by the complement of the base occupying the polymorphic site in the second allelic form.

If such an array is hybridized to a target sample containing heterozygous first and second allelic forms, the aggregate of the above two hybridization patterns is observed. That is, all probes in the first probe set from both the first and second group show perfect hybridization (albeit with reduced intensity relative to a homozygous target), and one additional probe from the second, third or fourth probe set in each group shows perfect hybridization. In each group, this probe has an interrogation position aligned with the polymorphic site and occupied by a base occupying the polymorphic site in one or other of the allelic forms.

Typically, arrays for analyzing precharacterized polymorphisms contain multiple subsets of each of the probe sets described, with a separate subset for each polymorphism. Thus, for example, a secondary array for analyzing a thousand polymorphisms might contain first and second groups of probes, each containing four probe sets, with each of the four probe sets, being divided into 1000 subsets corresponding to the 1000 different polymorphisms. In this situation, analysis of the hybridization patterns from four subsets relating to any given polymorphisms is independent of any other polymorphism. Analysis of the hybridization pattern of such an array to a target sample indicates which polymorphic form is present at some or all of the polymorphic sites represented on an array. Thus, the individual is characterized with a polymorphic profile representing allelic variants present at a substantial collection of polymorphic sites.

Methods for using arrays of probes for monitoring expression of mRNA populations are described in PCT/US96/143839, WO 97/17317, and U.S. Pat. No. 5,800,992. Some methods employ arrays having nucleic acid probes designed to be complementary to known mRNA sequences. mRNA populations or nucleic acids derived therefrom are applied to such an array, and targets of interest are identified, and optionally, quantified from the extent of specific binding to complementary probes. Optionally, binding of target to probes known to be mismatched with the target can be used as a measure of background nonspecific binding and subtracted from specific binding of target to complementary probes. Some methods employ arrays of random or arbitrary probes (also known as generic arrays). Such probes hybridize to complementary mRNA sequences present in a population, and are particularly useful for identifying and characterizing hitherto unknown mRNA species.

Arrays of probes immobilized on supports can be synthesized by various methods. Methods of forming arrays of nucleic acids, peptides and other polymer sequences are disclosed in, for example, 5,143,854, 5,252,743, 5,384,261, 5,405,783, 5,424,186, 5,429,807, 5,445,943, 5,510,270, 5,677,195, 5,571,639, 6,040,138, all incorporated herein by reference for all purposes. The oligonucleotide array can be synthesized on a solid substrate by a variety of methods, including light-directed chemical coupling, and mechanically directed coupling. See U.S. Pat. No. 5,143,854, WO 90/15070) and Fodor et al., WO 92/10092 and WO 93/09668 and U.S. Pat. No. 5,677,195, 6,040,193, and 5,831,070, U.S. Ser. No. 60/203,418, McGall et al., U.S. Ser. No. 08/445,332; U.S. Pat. No. 5,143,854; EP 476,014). Such arrays typically have at least 1000, 10,000, 100,000 or 1,000,000 different probes occupying 1000 different regions within a square centimeter. Algorithms for design of masks to reduce the number of synthesis cycles are described by Hubbel et al., U.S. Pat. Nos. 5,571,639 and 5,593,839. Arrays also can be synthesized in a combinatorial fashion by delivering monomers to cells of a support by mechanically constrained flowpaths. See Winkler et al., EP 624,059. Arrays also can be synthesized by spotting monomers reagents on to a support using an ink jet printer. See id.; Pease et al., EP 728,520. Arrays also can be synthesized by spotting preformed nucleic acid probes on to a substrate, as described by Winkler et al., EP 624,059. Such nucleic acid can be covalently attached or attached via noncovalent linkage, such as biotin-avidin or biotin-streptavidin. Alternatively, the DNA can be held in place by coating the surface of an array with polylysine, which is positively charged and binds to negatively charged DNA. Nucleic acid probe arrays of standard or customized types are also commercially available from Affymetrix, Inc. (Santa Clara, Calif.).

After hybridization of control and target samples to an array containing one or more probe sets as described above and optional washing to remove unbound and nonspecifically bound probe, the hybridization intensity for the respective samples is determined for each probe in the array. For fluorescent labels, hybridization intensity can be determined by, for example, a scanning confocal microscope in photon counting mode. Appropriate scanning devices are described by e.g., Trulson et al., U.S. Pat. No. 5,578,832; Stern et al., U.S. Pat. No. 5,631,734. Such devices are commercially available from Affymetrix, Inc. (Santa Clara, Calif.).

Reference sequences for polymorphic site identification are often obtained from computer databases such as Genbank, the Stanford Genome Center, The Institute for Genome Research and the Whitehead Institute. The latter databases are available at http://www-genome.wi.mit.edu; http://shgc.stanford.edu and http://ww.tigr.org. A reference sequence can vary in length from 5 bases to 100,000, 1 Mb, 10 Mb, 100 Mb or 1 GB bases. Reference sequences can be genomic DNA or episomes. In some methods, reference sequences are mRNA.

As discussed supra, the nucleic acid samples hybridized to arrays can be genomic DNA, cloned DNA, RNA or cDNA. Also, nucleic acid samples can be subject to amplification before or after enrichment. An individual genomic DNA segment from the same genomic location as a designated reference sequence can be amplified by using primers flanking the reference sequence. Multiple genomic segments corresponding to multiple reference sequences can be prepared by multiplex amplification including primer pairs flanking each reference sequence in the amplification mix. Alternatively, the entire genome can be amplified using random primers (typically hexamers) (see Barrett et al., Nucleic Acids Research 23, 3488-3492 (1995)) or by fragmentation and reassembly (see, e.g., Stemmer et al., Gene 164, 49-53 (1995)). Genomic DNA can be obtained from virtually any tissue source (other than pure red blood cells). For example, convenient tissue samples include whole blood, semen, saliva, tears, urine, fecal material, sweat, buccal, skin and hair. RNA samples are also often subject to amplification. In this case amplification is typically preceded by reverse transcription. Amplification of all expressed mRNA can be performed, for example, as described by commonly owned WO 96/14839 and WO 97/01603

The PCR method of amplification is described in PCR Technology: Principles and Applications for DNA Amplification (ed. H. A. Erlich, Freeman Press, NY, N.Y., 1992); PCR Protocols: A Guide to Methods and Applications (eds. Innis, et al., Academic Press, San Diego, Calif., 1990); Mattila et al., Nucleic Acids Res. 19, 4967 (1991); Eckert et al., PCR Methods and Applications 1, 17 (1991); PCR (eds. McPherson et al., IRL Press, Oxford); and U.S. Pat. No. 4,683,202, each of which is incorporated by reference for all purposes. Nucleic acids in a target sample can be labelled in the course of amplification by inclusion of one or more labelled nucleotides in the amplification mix. Labels can also be attached to amplification products after amplification e.g., by end-labelling. The amplification product can be RNA or DNA depending on the enzyme and substrates used in the amplification reaction.

Other suitable amplification methods include the ligase chain reaction (LCR) (see Wu and Wallace, Genomics 4, 560 (1989), Landegren et al., Science 241, 1077 (1988), transcription amplification (Kwoh et al., Proc. Natl. Acad. Sci. USA 86, 1173 (1989)), and self-sustained sequence replication (Guatelli et al., Proc. Nat. Acad. Sci. USA, 87, 1874 (1990)) and nucleic acid based sequence amplification (NASBA). The latter two amplification methods involve isothermal reactions based on isothermal transcription, which produce both single stranded RNA (ssRNA) and double stranded DNA (dsDNA) as the amplification products in a ratio of about 30 or 100 to 1, respectively.

There are many applications for the methods of the present invention. For example, one can apply the methods of the present invention to association studies and diagnosis of disease. The polymorphic profile of an individual may contribute to phenotype of the individual in different ways. Some polymorphisms occur within a protein coding sequence and contribute to phenotype by affecting protein structure. The effect may be neutral, beneficial or detrimental, or both beneficial and detrimental, depending on the circumstances. For example, a heterozygous sickle cell mutation confers resistance to malaria, but a homozygous sickle cell mutation is usually lethal. Other polymorphisms occur in noncoding regions but may exert phenotypic effects indirectly via influence on replication, transcription, and translation. A single polymorphism may affect more than one phenotypic trait. Likewise, a single phenotypic trait may be affected by polymorphisms in different genes. Further, some polymorphisms predispose an individual to a distinct mutation that is causally related to a certain phenotype.

Phenotypic traits include diseases that have known but hitherto unmapped genetic components (e.g., agammaglobulimenia, diabetes insipidus, Lesch-Nyhan syndrome, muscular dystrophy, Wiskott-Aldrich syndrome, Fabry's disease, familial hypercholesterolemia, polycystic kidney disease, hereditary spherocytosis, von Willebrand's disease, tuberous sclerosis, hereditary hemorrhagic telangiectasia, familial colonic polyposis, Ehlers-Danlos syndrome, osteogenesis imperfecta, and acute intermittent porphyria). Phenotypic traits also include symptoms of, or susceptibility to, multifactorial diseases of which a component is, or may be, genetic, such as autoimmune diseases, inflammation, cancer, diseases of the nervous system, and infection by pathogenic microorganisms. Some examples of autoimmune diseases include rheumatoid arthritis, multiple sclerosis, diabetes (insulin-dependent and non-independent), systemic lupus erythematosus and Graves disease. Some examples of cancers include cancers of the bladder, brain, breast, colon, esophagus, kidney, leukemia, liver, lung, oral cavity, ovary, pancreas, prostate, skin, stomach and uterus. Phenotypic traits also include characteristics such as longevity, appearance (e.g., baldness, obesity), strength, speed, endurance, fertility, and susceptibility or receptivity to particular drugs or therapeutic treatments.

Correlation is performed for a population of individuals who have been tested for the presence or absence of one or more phenotypic traits of interest and for polymorphic profile. The alleles of each polymorphism in the profile are then reviewed to determine whether the presence or absence of a particular allele is associated with the trait of interest. Correlation can be performed by standard statistical methods such as a K-squared test and statistically significant correlations between polymorphic form(s) and phenotypic characteristics are noted. For example, it might be found that the presence of allele A1 at polymorphism A correlates with heart disease. As a further example, it might be found that the combined presence of allele A1 at polymorphism A and allele B1 at polymorphism B correlates with increased risk of cancer.

Such correlations can be exploited in several ways. In the case of a strong correlation between a set of one or more polymorphic forms and a disease for which treatment is available, detection of the polymorphic form set in a human or animal patient may justify immediate administration of treatment, or at least the institution of regular monitoring of the patient. Detection of a polymorphic form(s) correlated with serious disease in a couple contemplating a family may also be valuable to the couple in their reproductive decisions. For example, the female partner might elect to undergo in vitro fertilization to avoid the possibility of transmitting such a polymorphism from her husband to her offspring. In the case of a weaker, but still statistically significant correlation between a polymorphic set and human disease, immediate therapeutic intervention or monitoring may not be justified. Nevertheless, the patient can be motivated to begin simple life-style changes (e.g., diet, exercise) that can be accomplished at little cost to the patient but confer potential benefits in reducing the risk of conditions to which the patient may have increased susceptibility by virtue of variant alleles. Identification of a polymorphic profile in a patient that correlates with enhanced receptiveness to one of several treatment regimes for a disease indicates that this treatment regime should be followed.

For animals and plants, correlations between polymorphic profiles and phenotype are useful for breeding for desired characteristics. For example, Beitz et al., U.S. Pat. No. 5,292,639 discuss use of bovine mitochondrial polymorphisms in a breeding program to improve milk production in cows.

Another application of the present invention is in the field of forensics. Determination of which polymorphic forms occupy a set of polymorphic sites in an individual identifies a set of polymorphic forms that distinguishes the individual. See generally, National Research Council, The Evaluation of Forensic DNA Evidence (Eds. Pollard et al., National Academy Press, DC, 1996). The more sites that are analyzed the lower the probability that the set of polymorphic forms in one individual is the same as that in an unrelated individual.

The capacity to identify a distinguishing or unique set of forensic markers in an individual is useful for forensic analysis. For example, one can determine whether a blood sample from a suspect matches a blood or other tissue sample from a crime scene by determining whether the set of polymorphic forms occupying selected polymorphic sites is the same in the suspect and the sample. If the set of polymorphic markers does not match between a suspect and a sample, it can be concluded (barring experimental error) that the suspect was not the source of the sample. If the set of markers does match, one can conclude that the DNA from the suspect is consistent with that found at the crime scene. If frequencies of the polymorphic forms at the loci tested have been determined (e.g., by analysis of a suitable population of individuals), one can perform a statistical analysis to determine the probability that a match of suspect and crime scene sample would occur by chance. If several polymorphic loci are tested, the cumulative probability of non-identity for random individuals becomes very high (e.g., one billion to one). Such probabilities can be taken into account together with other evidence in determining the guilt or innocence of the suspect.

An additional application of the methods of the present invention is the field of paternity testing. Paternity testing investigates whether the part of the child's genotype not attributable to the mother is consistent with that of the putative father. Paternity testing can be performed by analyzing sets of polymorphisms in the putative father and the child. If the set of polymorphisms in the child attributable to the father does not match the putative father, it can be concluded, barring experimental error, that the putative father is not the biological father. If the set of polymorphisms in the child attributable to the father does match the set of polymorphisms of the putative father, a statistical calculation can be performed to determine the probability of coincidental match. If several polymorphic loci are included in the analysis, the cumulative probability of exclusion of a random male is very high. This probability can be taken into account in assessing the liability of a putative father whose polymorphic marker set matches the child's polymorphic marker set attributable to his/her father.

An additional important application of the present invention is in the field of expression analysis. The quantitative monitoring of expression levels for large numbers of genes can prove valuable in elucidating gene function, exploring the causes and mechanisms of disease, and for the discovery of potential therapeutic and diagnostic targets. Expression monitoring can be used to monitor the expression (transcription) levels of nucleic acids whose expression is altered in a disease state. For example, a cancer can be characterized by the overexpression of a particular marker such as the HER2 (c-erbB-2/neu) protooncogene in the case of breast cancer.

Expression monitoring can be used to monitor expression of various genes in response to defined stimuli, such as a drug. This is especially useful in drug research if the end point description is a complex one; i.e., not simply asking if one particular gene is overexpressed or underexpressed. Therefore, when a disease state or the mode of action of a drug is not well characterized, the expression monitoring can allow rapid determination of the particularly relevant genes.

In arrays of random probes (sometimes known as generic arrays), the hybridization pattern is also a measure of the presence and abundance of relative mRNAs in a sample, though it is not immediately known which probes correspond to which mRNAs in the sample. However the lack of knowledge regarding the particular genes does not prevent identification of useful therapeutics. For example, if the hybridization pattern on a particular generic array for a healthy cell is known and is significantly different from the pattern for a diseased cell, then libraries of compounds can be screened for those that cause the pattern for a diseased cell to become like that for the healthy cell. This provides a detailed measure of the cellular response to a drug.

Generic arrays also can provide a powerful tool for gene discovery and for elucidating mechanisms underlying complex cellular responses to various stimuli. For example, generic arrays can be used for expression fingerprinting. Suppose it is found that the mRNA from a certain cell type displays a distinct overall hybridization pattern that is different under different conditions (e.g., when harboring mutations in particular genes, in a disease state). Then this pattern of expression (an expression fingerprint), if reproducible and clearly differentiable in the different cases can be used as a diagnostic. It is not required that the pattern be fully interpretable, but just that it is specific for a particular cell state (and preferably of diagnostic and/or prognostic relevance).

Both customized and generic arrays can be used in drug safety studies. For example, if one is making a new antibiotic, then it should not significantly affect the expression profile for mammalian cells. The hybridization pattern can be used as a detailed measure of the effect of a drug on cells, for example, as a toxicological screen.

The sequence information provided by the hybridization pattern of a generic array can be used to identify genes encoding mRNAs hybridized to an array. Such methods can be performed using DNA tags of the invention as the target nucleic acids described in WO 97/27317. DNA tags can be denatured forming first and second tag strands. The denatured first and second tag strands are then hybridized to the complementary regions of the probes, using standard conditions described in WO 97/27317. The hybridization pattern indicates which probes are complementary to tag strands in the sample. Comparison of the hybridization pattern of the two samples indicates which probes hybridize to tag strands that derive from mRNAs that are differentially expressed between the two samples. These probes are of particular interest, because they contain complementary sequence to mRNA species subject to differential expression. The sequence of such probes is known and can be compared with sequences in databases to determine the identity of the full-length mRNAs subject to differential expression provided that such mRNAs have previously been sequenced. Alternatively, the sequences of probes can be used to design hybridization probes or primers for cloning the differentially expressed mRNAs. The differentially expressed mRNAs are typically cloned from the sample in which the mRNA of interest was expressed at the highest level. In some methods, database comparisons or cloning is facilitated by provision of additional sequence information beyond that inferable from probe sequence by template dependent extension as described above.

### EXAMPLES

### Example 1

### Isolation of Cytoplasmic RNA from Tissue Culture Cells

In addition to using the methods of the present invention with cloned or genomic DNA, RNA may be used as a nucleic acid source for analysis. To prepare cytoplasmic RNA, cells were washed by adding 1 ml ice-cold PBS to a 10 cm tissue culture dish, and detaching the cells with a cell scraper. The cells were transferred to a 1.5 ml Eppendorf tube and centrifuged at 3000 rpm for 30 seconds. The supernatant was discarded and the cells were then suspended in 375 μl ice-cold lysis buffer ( 50 mM Tris-Cl, pH 8.0; 100 mM NaCl; 5 mM MgCl₂, and 0.5% (v/v) nonidet P-40) and incubated on ice for 5 minutes. The samples were then centrifuged, and the supernatants were removed and placed in clean tubes containing 8 μl 10% SDS. 2.5 μl of 20 mg/ml Proteinase K was then added to each tube and the samples were incubated at 37° C. for 15 minutes. 400 μl of phenol/chloroform/isoamyl alcohol was then added, the tubes were shaken, then centrifuged for 10 minutes at room temperature. The aqueous phase was removed, and the extraction was repeated. An additional extraction was done with 400 μl chloroform. Again, the aqueous layer was removed and the RNA was precipitated with 1 ml 100% ethanol and 40 μl 3M sodium acetate at pH 5.2. After precipitation, the pellets were rinsed with 1 ml 75% ethanol and 25% 0.1M sodium acetate, pH 5.2. Finally, the pellets were air dried and resuspended in 100 μl DEPC treated water. First strand cDNA synthesis was then carried out using the Life Technologies SuperScript II First Strand Synthesis kit (Life Technologies, Inc., Gaithersburg, Md.).

### Example 2

### Second Strand cDNA Synthesis and Adapter Ligation

Once RNA has been isolated, cDNA may be prepared to be used in the methods of the present invention. First, 4 μl 10×buffer (500 mM Tris-HCl pH 7.8, 50 mM MgCl₂, 100 μg BSA), 8 μl 0.4 mM dNTP, 20 μl first strand synthesis product, 2 μl DNA polymerase I (20 U/μl), 2 μl RNase H (4 U/μl), and water were combined and incubated at room temperature for one hour. Next, 10 μl 5×buffer, 0.25 μl DTT (100 mM) and 2 μl T4 DNA polymerase (10 U/μl) were added to the samples and incubated at 11° C. for 30 minutes. One volume of phenol-chloroform was then added, the tubes were centrifuged, and the upper layer was extracted with an equal volume of chloroform. The DNA was precipitated with 12.5 μl NaOAc (3M), 200 μl EtOH (100%), and 12.5 μl glycogen (500 μg/ml) and overnight incubation at −20° C. The DNA was then pelleted by centrifuging for 1 hour at 4° C., the pellet was washed with 500 μl of 70% ethanol, and resuspended in 23 μl of water.

The double-stranded, blunt-ended DNA products were then ligated to adapters by adding 2 μg of the DNA to 3 μl adapters (1 μg/μl), 3 μl 10×T4 DNA ligase buffer and T4 DNA ligase (400 U/μl) and incubating at room temperature overnight. The DNA products were purified through a Sephadex G-50 column and ethanol precipitated. Pellets were resuspended in buffer.

### Example 3

### Biotin Labeling of Target DNA

Biotinylated residues were incorporated into target DNA using nick translation. The target DNA can be DNA prepared by PCR amplification or a previously cloned DNA fragment, and other preparations known to those skilled in the art. The reactions were prepared by combining 1 μl purified DNA (0.1 mg/ml), 1 μl biotin 16-dUTP (0.04 mM), 2 μl 10×nick translation buffer (500 mM Tris-HCl (pH 7.5), 100 mM MgCl₂, 50 mM DTT), 1 μl dNTP mix (0.4 mM), [α-³²P]dCTP (3000 Ci/mmole), 1 μl DNAse I (10 mU), and water to 20 μl. The reaction mixture was incubated at 16° C. for 2 hours, then purified by spin column chromatography through Sephadex G-50 and ethanol precipitation. The pellet was resuspended in 10 μl buffer.

### Example 4

### Direct cDNA Selection (Primary Selection)

Repeat sequences in the cDNA were blocked. This was performed by combining 5 μl of human genomic Cₒt1 DNA (1 μg) with 5 μl of the linker-adapted cDNA (1 μg). The reaction mixture was overlayed with mineral oil and heated for 10 minutes at 100° C. The reaction was cooled to 65° C. and 10 μl of 2×hybridization solution ( 1.5 M NaCl, 40 mM Na phosphate buffer (pH 7.2), 10 mM EDTA (pH 8.0), 10×Denhardts solution, 0.2% SDS) was added to the reaction mixture under the oil. This mixture was then incubated for 4 hours at 65° C. After hybridization, 5 μl of biotinylated (50 ng) target DNA was denatured and combined with 20 μl of the blocked DNA and 5 μl of 2×hybridization solution ( 1.5 M NaCl, 40 mM Na phosphate buffer (pH 7.2), 10 mM EDTA (pH 8.0), 10×Denhardts solution, 0.2% SDS). This reaction was incubated for 2 days at 65° C.

### Example 5

### Strepavidin—coated Paramagnetic Bead Preparation

3 mg of beads were washed three times with 300 μl of strepavidin bead-binding buffer (10 mM Tris-HCl (pH 7, 5), 1 mM EDTA (pH 8.0), 1M NaCl) and the beads were resuspended in a final concentration of 10 mg/ml in the buffer. An aliquot of each labeling reaction was tested for the ability to bind the beads by combining 20 μl of the beads with 1 μl labeled DNA (10 ng/μl) and 29 μl bead binding buffer and incubating at room temperature for 15 minutes. The beads were removed by using a magnetic separator and transferred to a fresh tube. The radioactivity was then measured and the binding considered successful if the ratio of bound to free cpm was >8:1.

### Example 6

### Binding of Selected cDNA to Strepavidin—coated Paramagnetic Beads

The DNA was then captured by combining 50 μl strepavidin-coated beads, 30 μl of the annealed reaction mix and 50 μl strepavidin bead-binding buffer (10 mM Tris-HCl (pH 7, 5), 1 mM EDTA (pH 8.0), 1M NaCl). The mixture was incubated for 15 minutes at room temperature. The beads were removed using a magnetic separator and the supernatant was discarded. The beads were washed twice in 1 ml of 1×SSC/0.1% SDS at room temperature followed by three washes, 15 minutes each in 1 ml 0.1×SSC/0.1%SDS at 65° C. After the final wash, the beads were transferred to a fresh tube. Hybridized DNAs were eluted by adding 100 μl of 0.1M NaOH and incubating the reaction mixture for 10 minutes at room temperature. The mixture was desalted by spin-column chromatography through Sephadex G-50.

### Example 7

### Amplification of Selected DNAs

Three aliquots (1 μl, 5 μl and 10 μl) of eluted cDNA were combined with 5 μl primer (10 mM), 2.5 μl 10×amplification buffer, 2.5 μl dNTP mixture for PCR (2.5 mM), 0.2 μl Taq polymerase (5 U/μl) and water to bring the final volume to 25 μl. In addition, control reactions were set up. The negative control did not have the eluted DNA added, and the positive control added sample DNA that had not gone through the biotin labeling and selection steps. DNA was amplified using 30 cycles of denaturation at 94° C. for 30 seconds, annealing at 55° C. for 30 seconds and polymerization at 72° C. for 1 minute. Aliquots of the reaction products (0.5 μg/lane) were loaded onto a 1% agarose gel. Once the enrichment was confirmed, the amplification reaction was scaled up to yield at least 1.5 μg of selected DNAs. The pooled reactions were extracted with phenol:chloroform and the DNA was recovered by ethanol precipitation. The DNA was air dried and resuspended in buffer.

Secondary selection was carried out under the same conditions as the primary selection using 1 μg of selected DNA and 50 ng of target DNA. Repetitive sequences were blocked with 1 μg of the selected DNA being used in the reaction. The final amplification products were visualized on an agarose gel.

### Example 8

### Preparing Target DNA for Hybridization

After reducing sample complexity (and optionally labeling) target DNA was prepared for application to a chip as follows: 177 μl 5M TMACL, 3 μl 1M Tris (pH 7.8 or 8), 3 μl 1% triiton X-100, 3 μl 10 mg/ml herring sperm DNA, 3 μl 5 nM control oligo, and labeled DNA and H₂O to achieve a 300 μl final volume. In various embodiments, the concentration of labeled DNA ranged from about 0.1 pM to 100 pM. The samples were denatured at 99° C. for 5 minutes and spun down. The nucleic acid arrays were warmed to 50° C. about 20 minutes before adding the hybridization mixture. The sample nucleic acids were then added to a chamber containing the array, hybridized at 50° C. in a rotisserie using a rotation speed of 40 rpm.

### Example 9

### Staining and Scanning an Array

This example illustrates a procedure for detecting hybridization of sample to probes on an array.

Solutions

1. Streptavidin-phycoerythrin Solution

1 ml total (300 μl/chip)

470 μl water

500 μl 2×MES

20 μl acetylated BSA(50 mg/ml)

10μl streptavidin-phycoerythrin(1 mg/ml)

2. Antibody solution

1 ml total (300 ul/chip)

470 μl water

500 μl 2×MES

20 μl acetylated BSA(50 mg/ml)

10 μl biotinylated anti-streptavidin(1 mg/ml)

Procedures

First, a fluidics station (available from Affymetrix, Inc., Santa Clara) was primed with 6×SSPE/0.01% Triton X-100, and a scanner (also available from Affymetrix) was activated and an experimental information file was prepared according to the manufacturer's instructions. Hybridization solution was removed from the array and stored at −20° C. The array was then rinsed twice with 1×MES/0.01% Triton X-100, 300 μl streptavidin solution was added, and the arrays were incubated at room temperature for 20 minutes. The stain solution was then removed and the array was rinsed twice with 1×MES/0.01% Triton X-100. Next, 300 μl antibody solution was then added to the array and incubated at room temperature for 20 min. The antibody solution was removed and the array was rinsed twice with 1×MES/0.01% Triton X-100. 300 μl staining solution was again added to the array and incubated at room temperature for 20 min. The array was then inserted into the fluidics station and washed 6 times at 35° C. with 6×SSPE/0.01% Triton X-100. The array was then scanned.

### Example 10

### Fragmentation and Labeling of Genomic DNA or PCR Fragments

To fragment and label genomic DNA, the following reagents were combined: 30 μl of purified DNA sample (400 ng) and 3.7 μl of 10×buffer. Just before placing the sample into 37° C. water bath, 1 μl of 0.07 U DNaseI was added into the sample mixture (DNaseI dilution: 1.4 μl of DNaseI+18.6 μl cold 10 mM Tris, pH 8.0. Final concentration is 0.07 U/ul). The samples were mixed and incubated at 37° C. for 7 minutes. Next, the samples were heated at 99° C. for 10 min to inactivate the Dnasel, and then cooled on ice for 2 minutes. The samples were centrifuged at a maximum speed of 14,000 rpm for 20 seconds.

To label the fragmented DNA, 1 ul of TdT and 1 ul of biotin-ddATP were added to the fragmented DNA sample. The samples were mixed and centrifuged at a maximum of 14,000 rpm for 20 seconds. The samples were then incubated at 37° C. for 90 minutes and then at 99° C. for 10 minutes to inactivate the TdT enzyme. The samples were then cooled on ice for 2 minutes, centrifuged, and kept on ice until ready for hybridization.

An alternative procedure for fragmenting by DNaseI digestion and labeling that is particularly suitable for use with long range PCR products uses long range PCR products in a volume of 300-350 μl were obtained. The concentration of DNA was determined by OD₂₆₀ measurement. Next, 280 μg DNA was labelled to give a final target concentration of 5-10 pM for a complexity range of 3-6 MB. The labeling was performed in five independent Eppenderf tubes with each one containing 37 μl 10×One-Phor-All Buffer PLUS, 2 μl Gibco DNaseI (at 0.5 U/uL), 1 μl Dnase 1, purified LR-PCR products up to 330 μl in volume for a total reaction volume of 370 μl, each tube was incubated at 37° C. for 10 minutes, 99° C. for 10 minutes, and 25° C. for <5 minutes, and then spun briefly. 20 μl TdT (25 U/μl) and 20 μL biotin ddATP (1 mM) were then added to each tube, and then the tubes were incubated at 37° C. for 90 minutes, 99° C. for 10 minutes and 25° C. for <5 minutes.

### Example 11

### Removal of Repeat Sequences

In an alternative protocol to remove repeat sequences, human placenta DNA was digested with DNaseI as follows: 160 μg human placenta DNA (0.08 fM for the full length) was added to 220 μl reaction solution (64 μl DNA (2.5 ug/μl), 22 μl 10×buffer, 3.5 μl DNaseI (0.35 U), 132 μL wafer). 9 μl of 480 mM NaPO₄ buffer, pH 7.4 was then added to reach a final NaPO₄ concentration of 126 mM and a volume of 301 μl. The sample was denatured for 5 minutes at 99° C., incubated at 65° C. for 90 minutes to allow repeat sequences to hybridize, then diluted to 10 mM NaPO₄ for HPLC.

### Example 12

### HPLC Hydroxyapatite Chromatography

This protocol illustrates use of a hydroxyapatite column to separate single-stranded and double-stranded DNA. One application of this protocol used single-stranded fragments with an average length 60 bases from chromosome 21 and double-stranded fragments of herring sperm DNA (average length 500 bp). Both single- and double-stranded DNA were present at 9 μM. The column was an Econo-Pac CHT-II Cartridge having a DNA capacity of 160 μg. The column was loaded with DNA in 100 mM phosphate. At 10-20 mM phosphate hydroxyapatite binds both single and double stranded DNA. DNA was then eluted at a gradient from 10 mM to 1 M NaPO₄ buffer, pH 7.4 over 30 min. Elution was monitored by absorbance at 260 nm. At 5 minutes, there was a small peak indicating release of single stranded DNA, and at 25 minutes there was a larger peak indicating release of double stranded DNA, as shown in FIG. 1.

Additional methodology useful for practicing the invention are described in Birren et al. supra. All publications and patent applications cited above are incorporated by reference in their entirety for all purposes to the same extent as if each individual publication or patent application were specifically and individually indicated to be so incorporated by reference. Although the present invention has been described in some detail by way of illustration and example for purposes of clarity and understanding, it will be apparent that certain changes and modifications may be practiced within the scope of the appended claims.

What is claimed is:

1. A method of analyzing a subset of nucleic acids within a nucleic acid population, comprising:
(a) providing a population of nucleic acid fragments wherein at least some of said fragments have sequences that are repeated;
(b) denaturing said population of nucleic acid fragments;
(c) incubating said denatured population of nucleic acid fragments under conditions to produce a double-stranded subset of said population of nucleic acids and a single-stranded subset of said population of nucleic acids, wherein under said annealing conditions nucleic acid fragments of said population having repeat sequences preferentially anneal with each other relative to nucleic acid fragments of said population lacking repeat sequences;
(d) separating said single-stranded subset of said population of nucleic acid fragments from said double-stranded subset of said population of nucleic acid fragments;
(e) hybridizing said separated single-stranded subset of said population of nucleic acid fragments to probes on a nucleic acid probe array; and
(f) determining which of said probes on said array hybridize to said single-stranded subset of said population of nucleic acid fragments, thereby analyzing said single-stranded subset of said population of nucleic acid fragments.

2. The method of claim 1, wherein said population of nucleic acid fragments are genomic DNA fragments.

3. The method of claim 2, wherein said genomic DNA fragments are from a human genome.

4. The method of claim 3, wherein said DNA fragments from a human genome are fragments from a same chromosome of different human individuals.

5. The method of claim 1, wherein said separating step is performed by column chromatography.

6. The method of claim 5, wherein said column is a hydroxyapatite column.

7. The method of claim 6, wherein said separating step is performed under conditions whereby said single-stranded subset and said double-stranded are eluted in phosphate buffer.

8. The method of claim 1, wherein said separating step is performed by HPLC.

9. The method of claim 1, wherein said separating step is performed by successively performing hydroxyapatite chromatography and HPLC.

10. The method of claim 1, wherein said probe array comprises a set of probes complementary to a known reference sequence, said reference sequence being substantially identical to a sequence of said population of nucleic acid fragments.

11. The method of claim 10, wherein said population of nucleic acid fragments are from a chromosome from a first individual, and said reference sequences is a corresponding chromosome from a second individual.

12. The method of claim 10, wherein said population of nucleic acid fragments are genomic fragments from a first individual, and said reference sequences are genomic fragments from a second individual of a species closely related to said first individual.

13. The method of claim 10, wherein said population of nucleic acid fragments are genomic fragments from a non-human primate, and said reference sequence is from a human.

14. The method of claim 10, wherein said population of nucleic acid fragments are genomic fragments from a non-human mammal, and said reference sequence is from a human.

15. A method of analyzing a subset of nucleic acids within a nucleic acid population, comprising:
(a) providing a driver population of nucleic acids and a tester population of nucleic acids;
(b) denaturing said driver population of nucleic acids and said tester population of nucleic acids;
(c) annealing said driver population to said tester population to produce a single-stranded subset of nucleic acids and a double-stranded subset of nucleic acids;
(d) immobilizing said driver population of nucleic acids to produce an unimmobilized single-stranded tester subset of nucleic acids, an immobilized double-stranded tester-driver subset of nucleic acids and an immobilized single-stranded driver subset of nucleic acids;
(e) separating said unimmobilized single-stranded tester subset of nucleic acids from said immobilized double-stranded tester-driver subset of nucleic acids and said immobilized single-stranded driver subset of nucleic acids;
(f) hybridizing said unimmobilized single-stranded tester subset of nucleic acids to probes on a nucleic acid probe array; and
(g) determining which of said probes on said array hybridize to said unimmobilized single-stranded tester subset of nucleic acids, thereby analyzing said unimmobilized single-stranded tester subset of nucleic acids.

16. The method of claim 15, wherein said driver population of nucleic acids each bear a tag by which said driver population of nucleic acids can be immobilized to a binding moiety with affinity for said tag.

17. The method of claim 16, wherein said tag is biotin, and said binding moiety is avidin or streptavidin.

18. The method of claim 17, wherein said separating step is performed by immobilizing said immobilized double-stranded tester-driver subset of nucleic acids and said immobilized single-stranded driver subset of nucleic acids via said tags on said driver population.

19. The method of claim 15, wherein said driver population of nucleic acids are genomic DNA from a first source, and said tester population of nucleic acids are genomic DNA from a second source.

20. The method of claim 19, wherein said first source is from a tissue of a first species, and said second source is from a same tissue of a different species.

21. The method of claim 19, wherein said first source is from a first tissue of a first species, and said second source is from a different tissue of said first species.

22. The method of claim 15, wherein said immobilizing step is performed before said annealing step.

23. The method of claim 15, wherein said immobilizing step is performed before said denaturing step.

24. A method of analyzing a subset of nucleic acids within a nucleic acid population, comprising:
(a) providing a driver population of nucleic acids and a tester population of nucleic acids;
(b) denaturing said driver population of nucleic acids and said tester population of nucleic acids;
(c) annealing said driver population to said tester population to produce a single-stranded subset of nucleic acids and a double-stranded subset of nucleic acids;
(d) immobilizing said driver population of nucleic acids to produce an unimmobilized single-stranded tester subset of nucleic acids, an immobilized double-stranded tester-driver subset of nucleic acids and an immobilized single-stranded driver subset of nucleic acids;
(e) separating said unimmobilized single-stranded tester subset of nucleic acids from said immobilized double-stranded tester-driver subset of nucleic acids and said immobilized single-stranded driver subset of nucleic acids;
(f) dissociating said immobilized double-stranded tester-driver subset of nucleic acids to produce a subset of complementary tester nucleic acids and a subset of immobilized complementary driver nucleic acids;
(g) separating said subset of complementary tester nucleic acids from said subset of immobilized complementary driver nucleic acids;
(h) hybridizing said subset of complementary tester nucleic acids to probes on a nucleic acid probe array;
(i) determining which of said probes on said array hybridize to said subset of complementary tester nucleic acids, thereby analyzing said subset of complementary tester nucleic acids.

25. The method of claim 24, wherein said driver population is a population of genomic DNA fragments, and said tester population is mRNA or nucleic acids derived therefrom.

26. The method of claim 24, wherein said driver population is a population of genomic DNA fragments from a first source, and said tester population is genomic DNA from a second source.

27. The method of claim 26, wherein said tester population is from a genome of a first individual, and said driver population is from a genome of a different individual of a same species as said first individual.

28. The method of claim 26, wherein said tester population is from a genome of a first individual, and said driver population is from a genome of an individual of a different species than said first individual.

29. The method of claim 24, wherein either said driver population or said tester population or both said driver and said tester populations is a PCR amplification product.

30. The method of claim 24, wherein said driver population is from a plurality of noncontiguous regions of a genome of a species.

31. The method of claim 30, wherein said driver population is from at least ten noncontiguous regions.

32. The method of claim 24, wherein said driver population is mRNA or nucleic acids derived therefrom, and said tester population is genomic DNA.

33. The method of claim 24, wherein said driver population is mRNA or nucleic acids derived therefrom from a first source, and said tester population is mRNA or nucleic acids derived therefrom from a second source.

34. The method of claim 33, wherein said first source is from a tissue of a first species, and said second source is from a same tissue of a different species.

35. The method of claim 33, wherein said first source is from a first tissue of a first species, and said second source is from a different tissue of said first species.

36. The method of claim 24, wherein said immobilizing step is performed before said annealing step.

37. The method of claim 24, wherein said immobilizing step is performed before said first denaturing step.

38. The method of claim 24, wherein said driver population of nucleic acids each bear a tag by which said driver population can be immobilized to a binding moiety with affinity for said tag.

39. The method of claim 38, wherein said tag is biotin, and said binding moiety is avidin or streptavidin.

40. The method of claim 39, wherein said first separating step is performed by immobilizing said driver population of nucleic acids and tester population of nucleic acids hybridized to said driver population via said tags on said driver population.
